(19) United States
(12) Patent Application Publication
(10) Pub. No.: US 2004/0234971 A1
(43) Pub. Date: Nov. 25, 2004

(54) Diagnosis of pathogen infections using mass spectral analysis of immune system modulators in post-exposure biological samples

(76) Inventor: Joany Jackman, Walkersville, MD (US)

Correspondence Address: Albert J Fasulo II Office of Patent Counsel, The John Hopkins University, Applied Physics Laboratory, 1110 Johns Hopkins Road, Laurel, MD, 20723-6099, US

(21) Appl. No.: 10/470,890

(22) PCT Filed: Feb. 1, 2002
(86) PCT No.: PCT/US02/02956

Related U.S. Application Data
(60) Provisional application No. 60/265,797, filed on Feb. 1, 2001.

(51) Int. Cl. C12Q 1/68

(52) U.S. Cl. 435/6

(57) ABSTRACT

This proposal specifically addresses the problem of rapid medical evaluation of civilian or military personnel after potential exposure to biological weapons (BW). We propose to expand current non-invasive methods of infection detection through breath analysis used to measure nitric oxide (NO) production following exposure/infection. We will establish methods to analyze respired proteins as early markers of infection. Breath vapor will be collected and analyzed by mass spectrometry for indicators of host cell responses such as cytokine production and agent specific factors derived from biological threats such as cell wall components or virulence factors. As part of this effort we will develop the prototype for a single integrated collection device. This device would permit coordinate collection and analysis of NO levels and condensed exhaled air. Using collected exhaled air samples, we propose to develop a library of mass spectral patterns from cellular markers of infection using actual biological threat agents in vitro and in vivo. Initial work to detect, characterize and build a library based on the mass analysis of collected breath samples will be performed on the commercial matrix assisted laser desorption/ionization (MALDI) mass spectrometer IV instrument (Kratos). Subsequently r the portable MALDI known as the “Tiny TOF” will be used for this analysis. The “Tiny TOF”, a time of flight mass spectrometer (TOF) is currently under development at Johns Hopkins University/Applied Physics Laboratory (JHU/APL). Not only is the “Tiny TOF” capable of performing high resolution mass analysis of parent ions, but through the use of a novel design feature known as the curved field reflectron (CFR) this instrument will isolate product ions which result from laser fragmentation of the parent compound (known as post source decay (PSD). The fragmentation pattern produced by these product ions is considered to be an intrinsic property of an ionized molecule and can be used to “fingerprint” parent molecules which are difficult to resolve based on their molecular weight alone. These characteristic fragment patterns will be incorporated into the signature library to enhance the identification criteria of early warning markers of infection.

### CROSS-REFERENCE TO RELATED APPLICATIONS

This application claims the benefit of prior co-pending U.S. Provisional Application No. 60/265,797, filed on Feb. 1, 2001, the entire contents of which are hereby incorporated by reference. This application also hereby incorporates by reference the contents of a related U.S. application, filed on Feb. 1, 2002, entitled Mass Spectrometric Analysis of Complex Mixtures of Immune System Modulators, Jackman.

### BACKGROUND OF THE INVENTION

1. Field of the Invention

This invention is in the field of medical diagnosis of pathogen infections, based on mass spectrometric analysis of changes in expression of multiple immune system modulators like chemokines and cytokines in a biologic sample taken from a potential victim at various times following possible exposure to the pathogen.

2. Description of the Related Art

There is a great need for a rapid and sensitive method for diagnosing pathogen infections, especially in the aftermath following a real or suspected attack on military or civilian personnel with biological weapons, or a natural epidemic that has a short window of opportunity for effective treatment. Such an attack places an extreme burden on the medical system responsible for the rapid identification and treatment of infected persons. The medical community, civilian and military, must determine the best or most effective use of human pharmaceutical resources and hospitals following a bio-warfare attack. Current methods of diagnosing clinically infected persons are invasive, time-consuming, labor-intensive, or fail to offer predictive value for early stages of disease. Without proper early diagnosis, many people will face severe psychological torment, and will be treated unnecessarily with pharmaceuticals causing unnecessary side effects, and depleting the supply of drugs.

The outcome of any terrorist attack with real or “simulated” biological weapons can be considered successful if the attack results in stimulating panic or fear in the target population. Unlike the battlefield scenario where the ultimate purpose is to kill or debilitate large numbers of personnel, death is not necessarily the endpoint by which terrorists measure success. It is projected that a terrorist attack on a dense civilian population would rapidly overburden and effectively collapse regional emergency response and medical services.

The inability to distinguish those infected by pathogens disseminated from biological weapons, those infected by more benign agents, and those who simply perceive relevant signs and symptoms, is a major impediment to the rapid resolution of an attack with deadly biological weapons. The initial signs and symptoms reported for infection with biological weapons mimic those reported for more common illnesses such as the flu. An attack of this ilk would waste valuable resources and could have significant economic impact. One means to reduce or inactivate the threat posed by the use of biological weapons would be to quickly determine which victims were actually infected. This would limit the impact on emergency, law enforcement and medical services. At this time, there is no portable or even point-of-care system available for rapid, real time analysis to verify infection. A reliable system for analyzing early signs of infection would be one method for reducing the threat potential of the terrorist attack.

### SUMMARY OF THE INVENTION

The present invention provides a rapid, sensitive and affordable method for diagnosing exposure to one or more pathogens using mass spectrometric analysis of biological samples to detect the presence or absence of various immune modulators, the pattern of expression of which changes with exposure to pathogens. One method for diagnosing pathogen infection in a potentially infected animal using mass spectrometry, includes using a gridless mass spectrometer, determining a mass spectrometry library of patterns of expression for multiple molecular markers of infection in infected samples taken from an animal infected with a known pathogen, each pattern being associated with the known pathogen at a particular post-exposure time following exposure of the animal to the known pathogen; obtaining an analysis sample from a different animal potentially infected with the pathogen; placing the analysis sample on a substratum suitable for use in a mass spectrometer, saturating the analysis sample with matrix material, allowing the analysis sample to dry, processing the analysis sample in the gridless mass spectrometer to obtain a mass spectrograph for the analysis sample, and determining whether the mass spectrograph for the analysis sample includes a first pattern from the library of patterns. If it is determined that the mass spectrograph for the analysis sample includes the first pattern, then generating a diagnosis that the different animal is infected by the known pathogen. The method can also include putting in the library, patterns of expression for multiple molecular marker of infections in infected samples taken from multiple of animals infected with a respective plurality of known pathogens, each pattern being associated with a particular pathogen of the plurality of known pathogens at a particular post-exposure time following exposure of a respective animal of the plurality of animals to the particular pathogen; and where the known pathogen is a first pathogen of the plurality of known pathogens, which first pathogen is associated with the first pattern in the library of patterns. If it is determined that the mass spectrograph for the analysis sample includes the first pattern, then generating the diagnosis that the different animal is infected by the known pathogen at an respective infection time determined based on a particular post-exposure time associated with the first pattern match.

### BRIEF DESCRIPTION OF THE DRAWINGS

The present invention is illustrated by way of example, and not by way of limitation.

FIG. 1 is a graph illustrating Cytokine Detection in Complex Backgrounds by matrix assisted laser desorption/ionization (MALDI) time of flight (TOF), which shows the mass spectra of sputum (1A); sputum plus BSA (1B); equal amounts of three cytokines IL-6, TNF alpha and IL-1 alpha in a 1:1:1 ratio with a large excess of BSA in complex mixture of 1:1 sputum to cytokines (1C); and of the three cytokines IL-6, TNF alpha and IL-1 alpha in a 1:1:10 ratio with a large excess of BSA in a complex mixture of 1:1 sputum to cytokines (1D).

FIG. 2 is a graph illustrating the production of murine TNF-alpha in supernatants of cultured murine RAW 264.7 (RAW cells) macrophagocytic cells from lung at different post exposure times following infection with different bacteria and viruses.

FIG. 3, is a graph illustrating the production of murine IL-1 alpha in supernatants of cultured murine RAW 264.7 (RAW cells) macrophagocytic cells from lung at different post exposure times following infection with different bacteria and viruses.

FIG. 4 is a graph illustrating the production of murine IL-1 beta in supernatants of cultured murine RAW 264.7 (RAW cells) macrophagocytic cells from lung at different post exposure times following infection with different bacteria and viruses.

FIG. 5. is a photograph illustrating The Presence of Protein on Various Filters Suitable for use in mass spectrometers, including Durapore Modified PDVF, nitrocellulose, PVDF, and Hepa filter material.

### DETAILED DESCRIPTION

Recently, a reliable, affordable, quick and accurate method using gridless mass spectrometry to detect the presence of multiple immune system modulators and other infection markers, in a complex mixture such as a biological sample was discovered. This method is described in U.S. Provisional Application No. 60/265,797, filed on Feb. 1, 2001, and its descendant entitled Mass Spectrometric Analysis of Complex Mixtures of Immune System Modulators. The methods provide for detecting multiple immune system modulators and infection markers in a complex mixture by running a single sample through a gridless mass spectrometer without any surface preparation or preliminary purification of the sample, hereinafter, a “neat” sample. Immune system modulators that can be analyzed with the methods of the present invention include cytokines (interferons and interleukins), chemokines, virokines, host cell response molecules, peptide fragments, truncated receptors for immune modulators secreted by infected cells or pathogens. Infection markers like pathogen-encoded proteins and DNA fragments can also be thus analyzed.

The methods of the present invention for diagnosing pathogen infections are based on determining a library of mass spectral patterns of expression of immune system modulators in biological samples taken from animals known to be infected with known pathogens at various times after exposure and infection. The patterns in the library are compared to mass spectral patterns of expression of immune system modulators in the same type of biological samples taken from animals that may have been exposed and infected by a pathogen in the library. A diagnosis of pathogen infection is made if there is a match between a pattern from the potentially infected animal and a pattern in the library. Patterns of expression of any other molecules the secretion of which is known to be altered by exposure to a known pathogen can also be used in these diagnostic methods, including lipoxins, hydroxyeicosatraenoic acids, prostaglandins, leukotrienes, host cell response molecules, prions, virokines, peptide fragments, and truncated receptors for immune modulators secreted by infected cells or pathogens.

To obtain a positive diagnosis, the expression of molecular infection markers, especially cytokines and chemokines, in biological samples taken from an animal that may have been exposed to and infected with one or more pathogens (potentially infected animal samples), are collected at one or more times following the time of exposure and analyzed using mass spectrometry. Each of the mass spectral patterns in biological samples taken from the potentially infected animal is then compared to a library of mass spectral patterns of the same molecular infection markers in the same type of biological samples in “infected” samples taken from animals known to be infected with one or more known pathogens, which samples are taken at predetermined times after exposure and infection. A positive diagnosis for infection with a given pathogen is obtained if there is a match between the mass spectrograph pattern of expression of selected markers of infection in the sample taken from the potentially infected animal, and a pattern obtained from a known infected sample in the library of mass spectrograph patterns in biological samples taken from animals known to be infected with a known pathogen.

A practical rapid screening diagnostic method based on analyzing the expression of immune modulators such as cytokines in biological samples was not feasible until the discovery by Jackman, that gridless mass spectrometric analysis permitted the separation and clear resolution of more than one cytokine in a complex mixture such as a biological sample. Cytokines are nearly indistinguishable on SDS PAGE gels based on the relative molecular mobility at this level of resolution. The most widely used method for identifying cytokines in a complex mixture has been the Enzyme Linked Immunosorbent Assay (ELISA), which distinguishes cytokines antigenically (Balkwill, 1991). While these methods have been refined, they are expensive and time consuming. Where ELISAs are used, in some cases there is significant cross sensitivity between a given antibody to a cytokine and other closely related cytokines in the same class, for example among the interferons and interleukins, also ELISAs do not directly measure the mass of a molecule that reacts with an antibody. ELISA measures only that an antigenically related molecule is present. If more specific physical characterization of the molecule that is reacting with an antibody is needed than is obtained with an ELISA, a Western blot must be run to get relative molecular weight, which is very time consuming. In the early HIV tests that were run by ELISAs, a Western blot was required to confirm that the patient did not have another unrelated cross-reacting protein in his blood. It is important to note that the Elisa assay format takes about 90 minutes or longer to complete so that ELISAs do not give real-time analysis of cytokine expression, as does the use of mass spectrometric analysis. Also ELISAs can only analyze one molecule at a time, unlike the mass spectrometric analysis of a complex mixture that permits the detection of multiple of cytokines or other molecules at once.

In one embodiment, the biologic sample is exhaled breath condensate from lungs collected on a filter that traps the nonvolatile components including immune system modulators and infection markers. The filter with the protein on it is then put into a gridless mass spectrometer, using either an ultraviolet or infra red laser for analysis. Lungs are one of the first organs exposed to high concentrates of aerosol pathogens. Nearly all biological warfare attack scenarios involve aerosolization of pathogens to enable wide dissemination of materials. Because immune modulators are produced as part of the body's localized response to infection, lung secretions are one of the first places that changes in immune modulator expression will be detected. Bronchial lavage, tears, saliva and sputum may also be analyzed in a neat sample using the mass spectrometric methods described herein and in related applications. Analysis of urine by mass spectrometry may require washing the sample before applying it to the matrix for analysis. Blood can also be analyzed with some pre-preparation of the sample.

Cell Markers of Infection to be Used for Diagnosis

Candidate target markers of infection selected for diagnostic use in the present invention ideally have low or undetectable levels in healthy normal populations, but their concentration becomes elevated following infection. The change in levels of candidate molecules following exposure should be nearly immediate (less than 2 hours), robust, relatively long-lived (detectable for hours or days), and applicable to a wide distribution of the normal population following infection with biological agents. Ideally, equipment required for analysis and diagnosis is transportable for example by ambulance, and ultimately should be capable of miniaturization to be “man-portable” (<100 lbs). A portable diagnostic device would allow evaluation of an exposed population on site for defined early molecular responses to infection. It is most important that any diagnostic methods for detecting signs of infection should have a zero false negative rate and a low false positive rate.

Immune system modulators known to be involved in infectious or inflammatory processes include chemokines and cytokines (that include the interferons and interleukins). Cytokines are a varied group of proteins that are released by mammalian cells and act on other cells through specific receptors through which they elicit a wide variety of responses affecting the immune system. Cytokine actions include control of cell proliferation and differentiation, regulation of immune responses, hemopoiesis and inflammatory responses. Included among the cytokines are growth factors, interferons, interleukins and tumor necrosis factors. The majority of cytokines have molecular masses below 30 kilo Daltons in their monomeric form, and most have molecular masses in the ranges of 8,000-10,000 Daltons or 15,000-20,000 Daltons (Table 1). Chemokines are another immune system modulator that is a super family of soluble proteins implicated in a wide range of acute and inflammatory processes and other immunoregulatory functions. There are currently over 200 known proteins secreted in response to foreign body invasion and new candidates are added yearly (Callard, The cytokine fact book. Academic Press, New York, 1994; Vaddi, et al., The chemokine factbook. Academic Press, New York, 1997). Various arachidonic acid metabolites known collectively as eicosanoids, are also produced in large amounts in response to immune stress including pathogen infections (Reilly, et al., Bicosanoids and isoeicosanoids: indices of molecular function and oxidant stress. J Nutr 1998 February; 128(2 Suppl):434S-438S).

Previous studies have shown that the level of chemokines and cytokines and arachidonic acid metabolites in various biological samples (bronchial lavage fluid, exhaled condensate from lung, and blood changes following exposure to and infection by various pathogens. Therefore monitoring changes in chemokine and cytokine expression is a good diagnostic tool for detecting exposure to pathogens. While others have looked at expression of immune modulators in a host following infection, none have been able to collect and interpret patterns of expression of multiple immune system modulators in real time or as near real time as can be done with the present diagnostic method. The expression of other molecules is also known to change in response to pathogen infection and these molecules can also be included in the library of mass spectral patterns taken from infected samples. These other molecules include lipoxins, hydroxyeicosatraenoic acids, prostaglandins, leukotrienes, host cell response molecules, prions, virokines, peptide fragments, exhaled pathogen DNA, truncated receptors for immune modulators secreted by infected cells or pathogens, and any other molecules the secretion of which is known to be altered by exposure to a known pathogen. Table 1 gives the molecular weights, and other characteristics of various cytokines.

A number of events are initiated within the host following exposure to bacteria, foreign protein or a virus, to mount an immune response against the invading material. The events are initiated locally in the tissue at the actual site of invasion. By necessity, this response is immediate and robust.

TABLE 1
Characteristics of Known Inflammatory Cytokines*

| | Predicted | Observed | | |
|---|---|---|---|---|
| | MW | MW | Glycosylation | Immune Cell |
| Cytokine | in kDa | in kDA | (N, O—) | Source |
| TNFα | 26 | 17 | | T/M |
| IFNα | | 16-27 | N | T/M |
| IFNβ | | 20 | N | — |
| IFNK | 17.1 | 20, 25 | N | T |
| IL1I, θ | 17.5, 17.3 | 17-20 | N | M |
| IL2 | 15.4 | 15-20 | O | T |
| IL4 | 15 | 15-19 | N | T |
| IL6 | 20.8 | 26 | N | T/M |
| IL8 | 11.1 | 6-8 | — | M |
| MIP1I, θ | 8.6, 8.6 | 8-200, 7.8 | O | M/T |
| MIP2 (GROθ) | 11.4 | 7.9 | — | M |
| (GROK) | | | | |
| MCP1, 2, 3 | 8.7 | 8-18 | O | M |
| RANTES | 8 | 8 | O | T |
| GMCSF | 11.6 | 22 | N | T/M |

as compiled from Callard 1994 and Vaddi 1997.
*MIP = Macrophage inflammatory protein;
MCP = Monocyte chemoattractant protein;
GMCSF = Granulocyte/Macrophage Colony Stimulating Factor;
IL = Interleukin;
IFN = Interferon;
TNF = Tumor Necrosis Factor
**N = N linked; O linked
***M = Macrophage/Monocyte Derived; T = T-cell Derived

Cytokines are sub-grouped into pro-inflammatory (TH-1), anti-inflammatory (Th-2), and chemotactic cytokines (chemokines) (Kunkel, et al., Th-1 and Th-2 type cytokines regulate chemokine expression. Biol Signals. 5:197-202, 1996; Vaddi,1997). Cytokines are similar to hormones in that they are small, secreted proteins that have modulatory effects on other cell types, but they differ from hormones in that cytokine effects are exhibited locally (paracrine and autocrine) rather than at a distance (endocrine). Table 2.

TABLE 2
Cytokines Vs. Hormones

| | |
|---|---|
| Acts locally | Acts at a distance |
| Produced by many cell types | Produced by specialized cells |
| (immune, epithelial, etc.) | (thyroid, adrenal, etc.) |
| Synthesized transiently | Synthesized constitutively |
| following activation | following activation |
| Usually inactive in serum or plasma | Active in serum or plasma |
| Diffuses from tissues slowly | Easily measured in blood and can |
| forming | be correlated with physiological |
| | activity. |

Not all cytokines are expressed in each tissue type suggesting that there may be some spatially restrictive pattern to their expression based on the site of infection (Callard, 1994). The majority of cytokines have molecular weights below 30,000 Daltons in their monomeric form and most have molecular weights in the ranges of 8,000-10,000 Daltons or 15,000-27,000 Daltons (Table 1). The methods of the present invention will detect both monomers and dimers of cytokines or other immune system modulators.

Changes in Cytokines as a Result of Infection

The main route of entry of airborne pathogens into the body, and hence the organ first to be infected, is typically the lung. Changes in cytokines expression in response to pathogen infection were initially studied in whole lung tissue (Staniford, Expression and regulation of chemokines in acute bacterial Pneumonia. Biol Signals. 5:203-208, 1996a, Staniford, et al., Expression and regulation of chemokines in bacterial pneumonia. J Leukoc Biol. 59:24-28, 1996b; Ramshaw, et al., Cytokines and immunity to viral infections. Immunol. Reviews 159:119-135, 1997). More recently the appearance of diagnostic changes in the levels of cytokines shortly after pathogen infection has been measured by immunologic assays including western blots, immunoprecipitation and ELISA in bronchial lavage fluids (Lukas, et al., Activation and regulation of chemokines in allergic airway inflammation. J Leukoc Biol. 59:13-17, 1996; Fong, et al., The acute splanchic and peripheral tissue metabolic response to endotoxin in man. J. Clin. Invest. 85:1896-1904, 1989), induced sputum, (Pavrod, et al., The use of induced sputum to investigate airway inflammation. Thorax. 52:498-501, 1997; Pizzichini, et al., Indices of airway inflammation in induced sputum: reproducibility and validity of cell and fluid-phase measurements. Am J Pespir Crit Care Med. 154:308-317, 1996; Fahy, et al., Molecular and biochemical analysis of induced sputum from asthmatic and from healthy subjects. Am Rev Respir Dis. 147:1126-1131, 1993), and in condensed exhaled breath (Winsel, et al., Leukotrienes in breathing condensate released during bronchial challenge test. European Respiratory Journal (supplement):473, 1995).

It is generally accepted that the first cytokine to be produced following infection is TNFα (tumor necrosis factor). TNF alpha is secreted in the lungs by alveolar macrophages and T cells (Kranhbuhl, et al., Role of mycobacterial constituents in regulation of macrophage effector function in Virulence Mechanism of Bacterial Pathogens, ed. JA Roth et al., American Society for Microbiology, Washington D.C., 97-114, 1995). In non-human primates, bacterial lipopolysaccharide (LPS) derived from the cell walls of gram-negative bacteria has been shown to stimulate a 15-fold increase in TNF alpha measured in the blood using ELISA, a response that peaks at 2 hours and returns to baseline after about 4 hours. LPS is any of a group of related components of the outer leaflet of the outer membrane of the cell wall of gram-negative bacteria. During chronic bacterial pneumonia infection in mice, TNFα and IL-1 are produced in lung, and levels remain elevated for weeks following infection (Staniford, et al., Cytokines in host defense against pneumonia. J Investig Med. 45:335-345, 1997).

Unfortunately, respirable particulates such as coal dust can induce TNFα to nearly the same levels as LPS (Driscoll, et al., Cytokines and particle-induced inflammatory cell recruitment. Environmental Health perspectives. 105:1159-1164, 1997). Where there has been extensive damage to buildings coincident with exposure to airborne pathogens, such as may occur in a terrorist attack, it may be necessary to have a baseline for TNF alpha induced by particulate contamination to make an accurate diagnosis, or to rely on changes in the patterns of other molecular markers of infection. Analyzing changes in expression of more than one cytokine or molecular marker of infection over time dramatically increases the accurateness of the diagnosis and is readily accomplished using mass spectrometry.

Typically, IL-1 (interleukin) rises in the circulation within 4 hours, followed by the production of IL-6 after 6-8 hours. IL-6 production is sustained for a longer period of time than IL-1, and it is generally believed that IL-6 levels reflect of the severity of disease with high levels of serum IL-6 being associated with a fatal outcome (Waage, et al., The complex pattern of cytokines in serum from patients with meningococcal spetic shock: association between interleukin-1, interleukin-6 and fatal outcome. J. Exp. Med. 169:333-338, 1989). IL-6 is not expressed in uninfected animals. This cascade of TNF alpha and IL-6 production has been documented to occur in both humans (Michie, et al., Detection of circulation tumor necrosis factor after endotoxin administration. NE J. Med. 318:1481-1486, 1988) and mice (Fong, 1989; Staniford, 1996a, 1996b), with the exception that serum IL-1 was previously undetectable in humans even using ELISA.

Pro-inflammatory TH-1 cytokines (IL-2, IFNγ (interferon), GM-CSF (granulocyte/macrophage colony stimulating factor)) and anti-inflammatory TH-2 cytokines (IL-4, IL-5, Il-10) are all elevated in the lungs during the course of the bacterial infections; with exposure to Staphylococcus Enterotoxin B, changes occur in a matter of minutes. Anti-inflammatory TH-2 cytokine levels begin to decrease coincident with the decrease in TNF alpha and the TH-1 inflammatory cytokines.

Viral infections increase the production of IFNs α, β and γ (Ramshaw, et al., Cytokines and immunity to viral infections. Immunol. Reviews 159:119-135, 1997), while bacterial infections tend only to induce IFN γ secretion (Krahnbuhl, et al., Role of mycobacterial constituents in regulation of macrophage effector function in Virulence Mechanism of Bacterial Pathogens, ed. JA Roth et al., American Society for Microbiology, Washington D.C., 97-114, 1995). Likewise, intravenous injection of toxins such as Staphylococcus epidermis B (SEB) into mice increases serum IL-2 to unusually high levels (Gonzalo, et al., Differential in vivo effects of a superantigen and an antibody targeted to the same T cell receptor. J. Immunology 152:1597-1608, 1994). Other molecules such as arachidonic acid metabolites (eiconsenoids) appear as patterns of related molecules in response to infection with toxins such as SEB (Boyle, et al., Method for simultaneous isolation and quantitation of platelet activating factor and multiple arachidonate metabolites from small samples: analysis of effects of Staphylococcus aureus enterotoxin B in mice. Anal Biochem 216(2):373-82, 1994). Hence, comparison of patterns of eicosenoid expression also can be used to diagnose exposure to certain toxins.

Chemokines are generally undetectable in the lungs of naïve animals or healthy humans (Fong, 1989; Krahnbuhl, 1995; Fahy, 1993). However, some chemokines were observed to be chronically elevated in humans with such diseases as chronic symptomatic asthma (Fahy, 1993; Keatings, et al., Late response to allergen is associated with increased concentrations of tumor necrosis factor- and IL-5 in induced sputum. J Allergy Clin Immunol. 99:693-698, 1997; Lukas, 1996), cystic fibrosis (Koller, et al., Cytokine concentrations in sputum from patients with cystic fibrosis and their relation to eosinophil activity. Am J Respir Crit Care Med. 155:1050-1054, 1997) and in smokers (Pizzichini, 1996). Chronically high background levels of chemokines or cytokines may complicate diagnosis using the present methods, therefore the technicians administering the test would need to ask each potential victim if he or she is a smoker, an asthmatic, has cystic fibrosis, an autoimmune disease, etc. because such chronic conditions may alter baseline expression of immune system modulators and thereby affect the diagnosis. It may be decided that if the adverse effects of therapy are acceptable, it would be best to treat potential victims who have immune system complications even without a positive diagnosis.

The present methods can also be used to monitor the presence of virokines in biological samples, which are virally encoded small proteins that are secreted by the infected host cell. These proteins are often highly homologous to host immune proteins but are typically smaller (therefore with unique mass and fragmentation signatures), and more powerful. Examples of virokines include viral secreted proteins that block components of the host complement system, act as serine protease inhibitors, function as chemokine and cytokine agonists or antagonists and contribute to the proliferation of infected cells. Some virokines called “viroceptors” are homologs of host receptor s for cytokines that cause the host to down-regulate the inflammatory response elicited during infection. (Smith et al., Expert Opin Biol Ther 2001, May;1(3):343-57; Kotwal, et al., Virokines: mediators of virus-host interaction and future immune system modulators in medicine, Arch Immunol Ther Exp (Warsz) 1999; 47(3):135-8.) Many virokines have already been identified and associated with specific viral infections such as pox viruses including Myxoma. Nash, et al., PubMed index 10399068.

Parasites are also reported to evade detection and destruction by manipulating the host's immune system, for example by secreting helminth pseudocytokines. The presence of virokines or pseudocytokines in a biological sample would have diagnostic significance for identifying specific virus or parasite, or at least to diagnose a positive virus or parasitic infection in the host. Compilations of mass spectra signatures of virokines, pseudocytokines, pathogen-encoded proteins, etc. will permit the diagnosis of infections with agents that produce these proteins or fragments thereof.

Proteins Including Cytokines are Present and Can Be Captured in Exhaled Gases

The lungs provide a convenient route of entry for airborne pathogens, and it is known that cytokines are produced by the lungs in response to pathogen infection. In the most preferred embodiment of the present invention, the pattern of expression of molecular infection markers is analyzed in exhaled condensate from lung taken at predetermined times following the potential exposure to pathogens by collecting breath from a potential victim. Collection of exhaled condensate from potential victims is quick and non-invasive. According to the present invention, the condensate is collected on a filter that is then inserted into the gridless mass spectrometer for analysis; the pattern of expression of molecular infection markers in the potentially infected sample is then compared against a library of patterns obtained from exhaled condensate taken from animals infected with known pathogens. A positive diagnosis is obtained if the pattern of expression of molecular infection markers in exhaled condensate in the sample from the potential victim matches the pattern of expression in exhaled condensate from an animal with a documented infection (the infected sample) by a known pathogen.

The diagnostic value of analyzing exhaled condensate depends on the detection and stability of the markers in the condensate. In a recent study, exhaled condensate was obtained from asthmatics was collected and frozen before and after a non-specific bronchial challenge test. The condensate was analyzed for the presence of leukotrienes B4, C4, D4, E4, and F4. The results showed a correlation between elevated leukotriene levels in the condensate and the degree of asthmatic disease. (Becher, et al., Breath condensate as a method of noninvasive assessment of inflammation mediators from the lower airways, Pneumologie 1997, Apr; 51 Suppl. 2:456-9.) Leukotrienes are a family of pharmacologically active substances derived from polyunsaturated fatty acids, notably arachidonic acid.

Exhaled breath condensate was also analyzed using ELISA in pigs following lung transplantation to monitor changes in IL-10 levels over time to determine if changes in IL-10 are an indication of ischemia-reperfusion injury. McRae et al., Detection of IL-10 in the exhaled breath condensate, plasma and tissue during ischemia-reperfusion injury in experimental lung transplantation, J. Heart and Lung Transplantation, February 2001, p. 184, Abstract No. 104.) Another recent study confirmed the presence of proteins and urea in exhaled breath condensate collected from both healthy humans and patients with COPD (chronic obstructive pulmonary airway disease). (Gessner et al., Pneumologie, 2001, Sep. 55(9):406-8.) Breath condensate volume increased linearly with time of collection as long as ventilation remained fairly constant.

In another study, between 4 micrograms and 1.4 milligrams of protein were collected from breath condensate and analyzed using polyacrylamide gel electrophoresis (PAGE). Proteins in breath originate partially from the naso-oropharyngeal tract and from lower regions of the airways and are presumed to be a reflection of secretions into the interstitial space. (Scheideler et al., Detection of nonvolatile macromolecules in breath. A possible diagnostic tool? Am Rev Respir Dis 1993;148(3):778-84.) In the same study, 13 patients suffering from various diseases of the respiratory tract were sampled and breath condensate had up to 370 picograms of Il-1 beta, 120 pg of TNF alpha and 2,159 U sIL-2R per ml. These studies are important in that they confirm the concept that non-volatile protein molecules including molecular markers of infection like cytokines are present in and can be concentrated from exhaled gases.

Mass Spectrometry

The introduction of the commercial matrix assisted laser desorption/ionization (MALDI) mass spectrometer instrument in 1988 provided a new and powerful technique for the analysis of large biological molecules, resulting in the commercialization of TOF (time of flight) mass spectrometers by a number of manufacturers (Noble, Anal. Chem. 67 497A-507A.1995; Karas, et al., Anal. Chem. 60: 2299, 1988; Tanaka, 1988). In the simple (linear) TOF mass spectrometer, ions are formed and accelerated in a short source region. The ions flight times (t) through a longer drift region (D) are measured to determine their mass (m) according to the equation:

$$t = {( \frac{m}{2 eV} )}^{1 / 2} D$$

where eV=kinetic energy (Cotter, Time-of-Flight Mass Spectrometry: Instrumentation and Applications in Biological Research, American Chemical Society, Washington, D.C. 1997). The laser driven ionization of the source material is improved by mixing the material with an energy-absorbing matrix. The matrix assists ionization of the source material by transferring energy, hence the name MALDI-TOF mass spectrometry.

Some mass spectrometers have a “reflectron” to reflect ions back towards a rear mounted detector, which effectively increases the flight tube length and thus improves resolution of the mass spectrograms. Focusing occurs as ions with higher kinetic energy are subjected to a greater reflecting voltage by passing deeper into the reflectron than those ions of the same molecular mass but lower kinetic energy. The reflectron helps to reduce the intrinsic heterogeneity of ion movement to the detector based on variations in initial kinetic energy.

Reflectron spectrometry may be used to detect cytokines if the reflectron is capable of reflecting the mass range that includes the mass of the cytokines in the sample. Reflectron spectrometry was not used to obtain any of the spectra in the FIG.s herein.

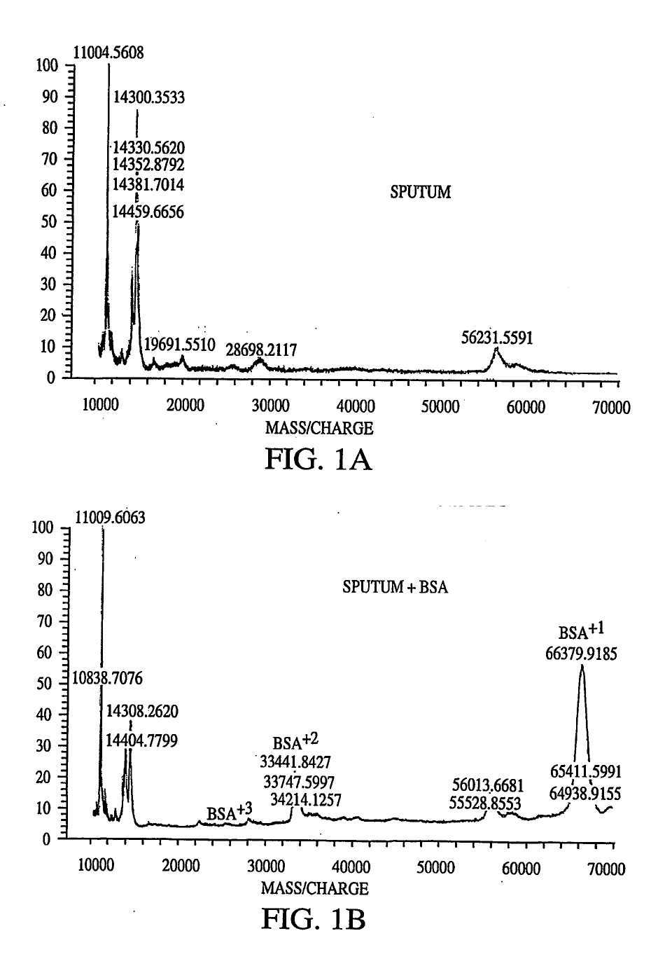

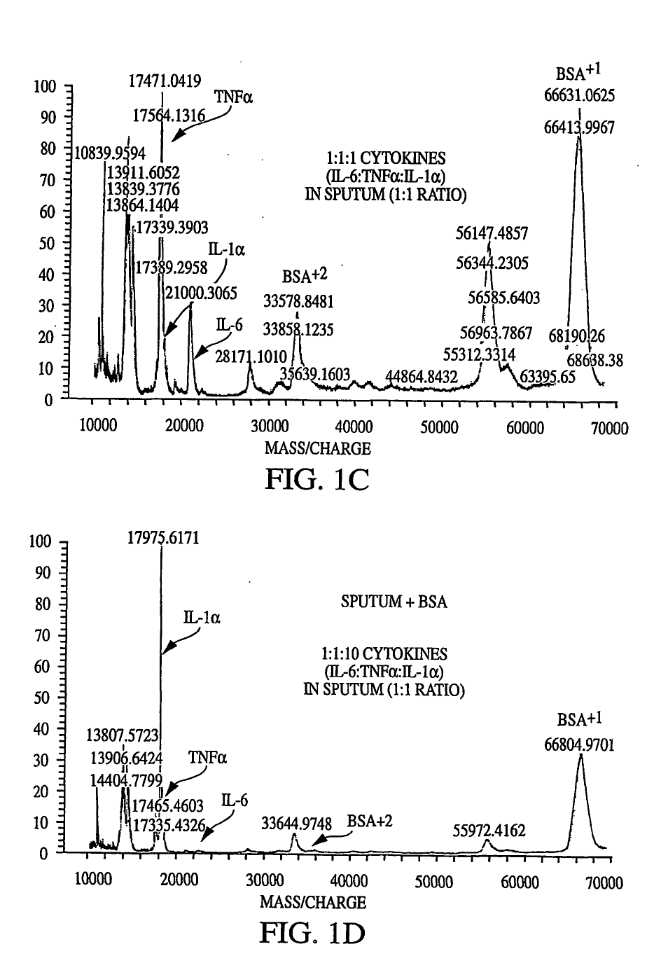

In the matrix assisted laser desorption/ionization (MALDI) mass spectrometer MALDI-TOF, low molecular mass compounds (less than about 30 kilo Daltons) generally fragment inside the ion source, providing significant structural information in the mass spectra obtained from either linear or reflectron analysis. Because the excess energy from the ionization process is absorbed by a larger number of covalent bonds, higher molecular mass compounds will fragment over a longer time frame, with much of the fragmentation occurring in the drift region. The fragmentation pattern of a molecular ion is believed to be an intrinsic property of the molecule, therefore it is a unique “signature” for the respective molecule. Known as Post Source Decay (PSD) in the art, the product ions resulting from fragmentation of a molecular ion in this region will have a range of flight times. The fragmentation pattern of a molecular ion is believed to be an intrinsic property of the molecule. PSD can therefore be used to distinguish molecules of similar molecular mass which cannot be resolved by simple linear ionization techniques and is generally best suited to molecules in the 6-10 kDa range such as those listed in Table 1 which includes cytokines (Cotter, 1997) In order to use PSD, the operator must know the pattern of cytokines or other immune system modulator that will be detected in the sample (or that he wishes to look for) before running the samples, because he must set a cut-off molecular mass to use this feature. PSD therefore optimizes the mass range desired. PSD is most advantageous for identifying and distinguishing among unique molecules that have the same molecular mass but different amino acid sequences, and that differ from one another by less than about 500 Daltons. PSD was not used to generate any of the spectra in FIG. 1 because it was not needed to identify the cytokines in any of the samples, whether the cytokines were suspended only in water or in sputum.

Mass Spectrometry can be Used to Identify More than One Closely Related Immune System Modulator at a Time in a Complex Mixture

One of the first cytokines known to be expressed following pathogen exposure is TNF alpha that is easily identified by its ionic mass. By contrast, TNF alpha is indistinguishable from IL-1 on SDS PAGE gels, and has previously been typically identified using ELISA assays that are time consuming and expensive, and that must be customized for each immune modulator or other molecule based on its antigenicity. In the event of an attack with biological weapons, there would not likely be enough antibody or equipment to screen large numbers of potentially infected people using ELISAs or other immune-based assays.

FIG. 1 shows that more than one cytokine in a complex mixture can be analyzed in a single sample using gridless mass spectrometry. All cytokines used in this experiment were recombinant human (rh) cytokines. FIG. 1 gives the mass spectra of sputum alone (1A); sputum plus BSA (1B), sputum plus equal amounts of three cytokines IL-6(rh), TNF alpha (rh) and IL-1 alpha (rh) in a 1:1:1 ratio with a large excess of BSA in complex mixture of 1:1 sputum: cytokines (1C); and of the three cytokines IL-6(rh), TNF alpha(rh) and IL-1 alpha (rh) in a 1:1:10 ratio with a large excess of BSA in complex mixture of 1:1 sputum to cytokines (1D). The scale of the signal intensity on the y axis was maximized to the signal intensity for the peak at about 17,000, which corresponds to the peak of TNF-alpha (rh). Mass spectrometry was performed using a Kratos AXIMA MALDI-TOF gridless mass spectrometer in linear mode with ultra violet lasers, with and without pulsed extraction. A matrix of sinapinic acid prepared in 70% acetonitrile HPLC grade) and 30% acidified water (HPLC Grade) was used.

All three cytokines and BSA could be distinguished in the mass spectral of these complex mixtures (1C and 1D). In 1C where the cytokine ratio of IL-6(rh), TNF alpha(rh) and IL-1 alpha(rh) was 1:1:1, the TNF-alpha(rh) peak is the highest and IL-6(rh) is the smallest. While IL-1 alpha(rh) peak is small, it is consistent with what is expected from the single cytokine spectra. In 4D where the cytokine ratio of IL-6(rh), TNF alpha(rh) and IL-1 alpha(rh) was 1:1:10, the IL-1 alpha(rh) peak is now the highest and the IL-6(rh) peak is now very small, but clearly detectable. To obtain a mass spectral analysis of more than one cytokine or immune modulator in a complex mixture, gridless mass spectrometer must be used.

Using the methods described in Jackman, Mass Spectrometric Analysis of Complex Mixtures of Immune System Modulators, multiple different cytokines in a complex mixture in neat biological samples could be identified using a gridless mass spectrometer. Resolution of more than one cytokine in a complex mixture can also be obtained using the reflectron mode and/or an infra red laser. Standard proteomic preparations for mass spectrometry typically involve harsh treatments that may have denatured the immune system modulators preventing their identification in a complex protein sample. Liquid chromatography separations that are often done to partially purify complex mixtures before mass spectrometric analysis, may remove some cytokines. Also, some of the treatments used in preparing proteomic samples modify glycosylated molecules, including any immune system modulators, which may preclude their identification in a complex mixture, in part because the mass spectrometer may not be able to supply enough energy to ionize the glycosylated molecules. Jackman discovered that resolution of one or more cytokine in a complex mixture could not be reliably obtained using an acceleration grid in the mass spectrometer. As reported, resolution and separation of immune modulators is improved when an Einzel lens is used. An Einzel lens is an electronic lens that focuses ions on to a narrow path down the flight tube of the mass spectrometer, creating less static and producing a more distinct peak in the final mass spectrogram.

All mass spectra have a certain amount of noise in the signal along the base-line on the x-axis. Small peaks can be reliably distinguished from background noise if the height of the peak is at least 2.5 times greater than the local background noise. For comparison purposes, reliable mass spectra for known samples of pure compounds can be compiled using the MALDI mass spectrometer to obtain “gold standard” spectra for comparison with other mass spectrometry methods. MALDI mass spectrometers using either infrared lasers or ultra violet can be used. Routine experimentation using known standards will determine the appropriate matrix for the immune system modulator or infection marker being analyzed, and whether post source decay analysis (PSD) is advantageous to obtain sufficient resolution of the peaks for diagnostic purposes. Sinapinic acid prepared in 70% acetonitrile (HPLC grade) and 30% acidified water (HPLC Grade) was the preferred matrix for analyzing cytokines using mass spectrometry. New infra red mass spectrometers have infra red lasers that can be tuned to different wavelengths to improve resolution. For some of the new instruments, a matrix of water may be suitable for analysis.

Routine experimentation will also determine whether pulsed or nonpulsed extraction should be used. Pulsed extraction must be set to optimize for a particular size and mass range. Pulsed extraction can improve separation if the operator knows which cytokines are in the sample ahead of time. Pulsed extraction is therefore not recommended for screening samples for multiple immune system modulators because it will limit detection. However, if the operator is looking for a specific group of molecules that are close in size, pulsed extraction may be useful.

The fragmentation pattern produced by mass spectral analysis of parent ions, such as cytokines and other molecules, is an intrinsic property of an ionized molecule and can therefore be used to “fingerprint” parent molecules that may be difficult to resolve based on their molecular weight alone. These characteristic fragment patterns are used to create a signature library for each molecule to enhance the identification criteria of early warning markers of infection.

Changes in Cytokine Secretion from Lung Cells Infected with Pathogens

We have focused on measuring the local immune response that takes place in the lung following exposure to the type of agents which would be expected to be used in a biological weapons attack. Bacillus anthracis (BA), Yersinia pestis (YP), Venezuelan Equine Encephalitis (VEE) and Staphylococcus enterotoxins (SE Tx) are all effective biological weapons when delivered by an aerosol route. Since the initial mammalian host cell response to pathogen infection is typically local, we evaluated changes in the secretion of immune system modulators in order to detect the earliest signs of lung infection.

The host cell response to infection in the lung has been well documented for bacterial pneumonias and influenza (Staniford, 1996a, Staniford, 1996b; Ramshaw, 1997). Candidate markers of diagnosing infection must meet the following criteria to be useful early markers for diagnosis.

They must be secreted immediately.

Their production should be robust to provide the necessary amplification of signal required to detect low numbers of agents.

The production or stability of the marker must be relatively long-lived so that it can be assessed over a wide time frame following exposure.

The marker should be easily detectable in a complex sample because processing would severely slow down and, perhaps, confound the analysis.

The signal should be applicable to the normal population as a whole following infection with biological agents.

The secreted proteins produced by immune cells following infection appear to satisfy all these criteria. Known collectively as cytokines and chemokines, there are currently over 200 known proteins secreted in response to foreign body invasion and new candidates are added yearly (Callard, 1994; Vaddi, 1997). In addition, arachidonic acid metabolites known collectively as eicosanoids, are produced in large amounts and varied types in response to immune stress (Reilly, 1998), making them good candidates for including in the mass spectral analysis of biological samples. In one application of the present diagnostic method, the secretion of eicosenoids is analyzed in one or more biological samples from a patient being treated with antibiotics to monitor the efficacy of the drug therapy; eicosenoid levels should go down as the infection is brought under control.

In addition to immune modulators, samples of infected cells or biological samples from infected animals should be analyzed for the expression of pathogen-encoded molecules. These include virulence factors and invasion-associated proteins such as capsule (Bacillus anthracis) and F1 antigen (Yersinia pestis) which blind host immune systems to bacteria, virokines produced by some viruses to co-opt host responses (Smith, et al., Vaccinia virus immune evasion. Immunol. Reviews 159:137-154, 1997), protein toxins and bacterial waste products such as LPS or cell wall components and pathogen-encoded DNA. It is expected that new cytokines and pathogen-encoded markers with diagnostic potential will continue to be identified.

Most of the pathogens that are likely to be used for biological warfare are already known and are available (under tight restrictions) for research. Therefore, a library of mass spectral patterns in various biological samples from infected animals can be accumulated to determine the pattern of expression of cytokines, other immune modulators and pathogen-encoded molecules in vitro and/or in vivo at various times following exposure to known pathogens. Such a mass spectral library should be compiled and stored for future diagnostic purposes. As many variants of the pathogen as can be obtained should be tested because the pattern of immune modulator expression may change from one variant to another. It is especially important to have mass spectra expression libraries ready ahead of time because some of the deadly pathogens that may be used in biological warfare have very narrow windows of opportunity for effective treatment. For example, the disease anthrax (caused by Bacillus anthracis) is nearly always fatal unless it is treated in the first forty-eight hours. Plague caused by Yersinia pestis is also fatal in 1-4 days, especially where the bacteria is inhaled.

In most cases, samples of the pathogen(s) can be obtained from air, debris, clothes, etc. Samples of pathogens can be analyzed using various techniques to obtain a positive identification of the pathogen or pathogens that were released. The task of diagnosing which potential victims have been exposed to the pathogen(s) and which have not, is simplified if the identity of the pathogen(s) is known. Just because people were exposed to the pathogen does not mean that they are infected. This method permits detection of infection among an exposed population that includes people who are exposed but do not become infected.

Differences in Mass Spectral Patterns of Cytokine Expression at Various Times Following Exposure to Bacteria or Viruses Permit Differential Diagnosis

As shown above in FIG. 1, we have demonstrated the ability of gridless mass spectrometers to rapidly and clearly distinguish low molecular weight cytokines in a complex mixture (sputum) in single assay of a neat sample even where the mass of the cytokines is separated by as little as 500 Daltons.

These results show that mass spectrometry can detect the presence of more than one immune modulator at a time in complex biological samples such as sputum. By analogy to sputum, samples of saliva, bronchial lavage and exhaled breath condensate can also be similarly analyzed for the presence of multiple immune modulators in a single neat sample. These types of biological samples have similar phospholipid compositions, thus the feasibility of analyzing sputum without any preliminary preparations predicts the feasibility of analyzing bronchial lavage and exhaled breath condensate without any preliminary preparations. With only routine experimentation, other biological samples can also be analyzed using the methods of the present invention, including urine that typically requires washing before analysis, and blood.

To be useful in screening large numbers of potential victims in a short time with limited staff, the biological samples should be easy to obtain, and samples must be a repository for the secreted immune system modulators, pathogen-encoded proteins, host cell response molecules, etc. that are to be analyzed. In one preferred embodiment, the diagnostic methods of this invention are applied to analyze exhaled breath condensate, which has been shown to contain expelled proteins and immune system modulators.

In order to set up a diagnostic screening assay using mass spectrometric analysis of secreted cytokines, experiments were conducted on murine RAW 264.7 (RAW cells) macrophagocytic cells from lung in vitro. The release of cytokines into the supernatants from culture media for these cells is predictive of the presence of cytokines released into breath condensate, bronchial lavage, and sputum.

In the experiments described below, samples of supernatant taken from RAW cells was analyzed at various times following exposure to and infection by a known pathogen. Six important cytokines, the levels of which have been reported to change with infection, were analyzed in a single sample. They are IL-1 alpha, IL-1 beta, IL-2, IL-6, TNF alpha and Interferon gamma in neat samples. The identity of the cytokines was determined by comparison of the mass spectra of the experimental samples to mass spectra of purified murine cytokine controls and to ELISA assays of the samples using murine antibodies.

RAW cells were infected with one of the following: Bacterial agents: LPS, Yersinia Pseudotuberculosis, Yersinia kristensenii, Bacillus anthracis spores, Staphylococcus aureus (which produces Staphylococcus enterotoxin B), Staphylococcus epidermis, and Haemophilus influenza.

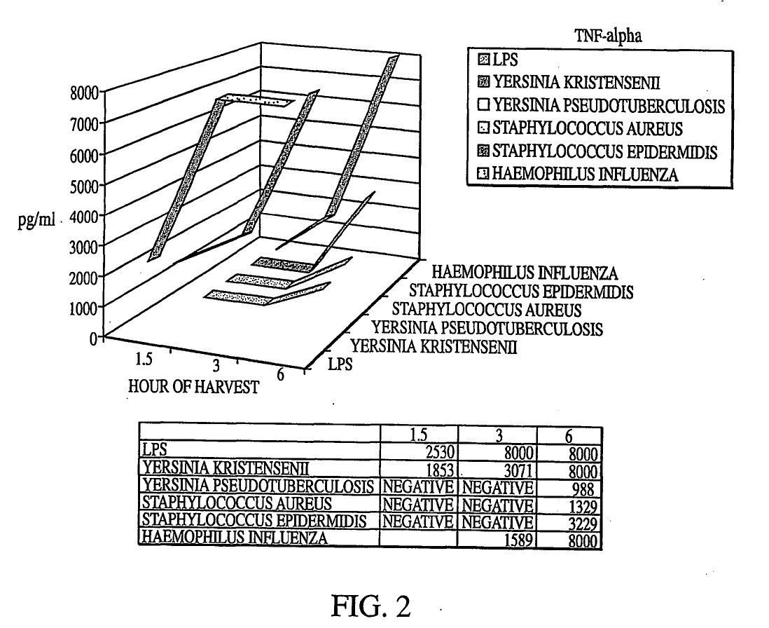

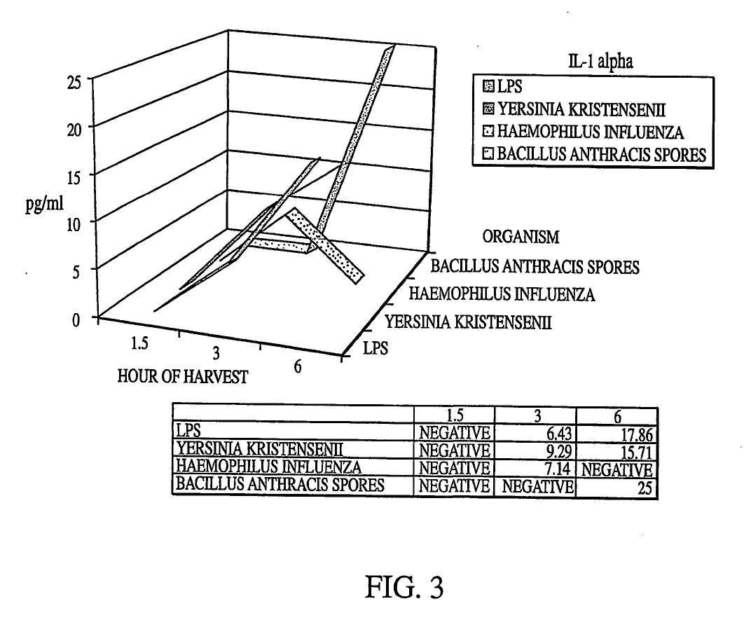

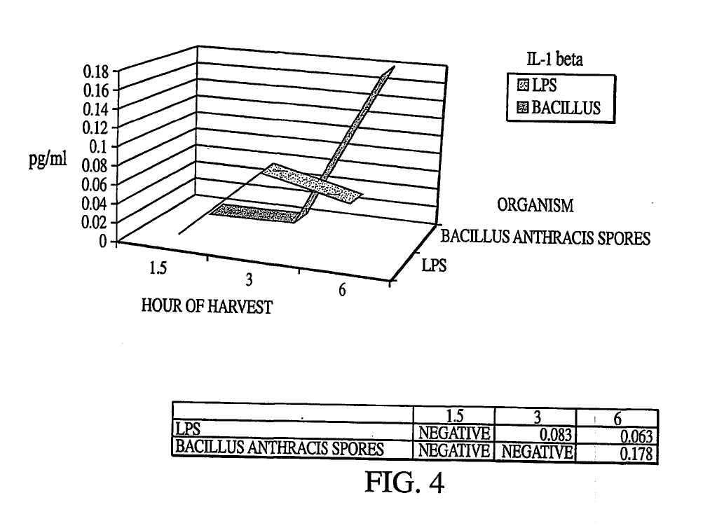

Bacteria and virus in amounts sufficient to infect the cultured cells were added to triplicate cultures. Supernatant was harvested by collection and centrifugation 0, 1.5, 3.0 and 6.0 hours following exposure. Mass spectrometry was performed using a KRATOS AXIMA MALDI-TOF gridless mass spectrometer in the linear mode, with and without pulsed extraction. A matrix of sinapinic acid prepared in 70% acetonitrile (HPLC grade) and 30% acidified water (HPLC Grade) was used. Routine experimentation will permit the investigator to optimize the matrix for different samples and molecular markers. The results are shown in Table 3 and in FIGS. 2, 3, and 4.

Cells were exposed to LPS as a control. LPS caused expression of IL-1 alpha, IL-1 beta, TNF-alpha and IL-6 at various times after exposure. By 3 hours after exposure to LPS, all four of these cytokines were expressed and expression continued to be detected at all time points tested through six hours after exposure. By contrast, unexposed cells expressed no cytokines at any time. Only Bacillus globigii spores failed to cause expression of at least one cytokine at one of the time points evaluated.

The results show that a positive diagnosis required comparison of patterns of expression of more than one immune modulator. Changes in expression of four immune modulators (IL-1 alpha, IL-1 beta, TNF-alpha and IL-6) permitted us to distinguish among all of the pathogens tested except Bacillus globigii spores, infection with which did not induce post-exposure expression of any of the six cytokines analyzed. Expression of two of the six cytokines tested (IFN-gamma and IL-2) was not induced by infection with any of the pathogens tested. Infection with Yersinia kristensenii (Yk) and Haemophilus influenza (Hi) both caused expression of IL-1 beta and TNF-alpha at 1.5, 3 and 6 hours. However, Hi infection could be diagnosed and distinguished from Yk infection because cultures exposed to Yk also induced the expression of IL-6 and IL-1 alpha at 6 hours post-exposure, while Hi did not. FIGS. 2, 3 and 4 show the pattern of expression of TNF-alpha, IL-1 alpha, and Il-1 beta, respectively, at various times following exposure to various bacteria and viruses indicated on the respective graphs.

The results in Table 3 also show that with Bacillus anthracis spore infection, TNF alpha is not elevated at any of the times tested, even at six hours after infection. This is unexpected because TNF alpha is typically the first cytokine expressed after pathogen infection. This observation may also have diagnostic significance for diagnosing anthrax.

It is anticipated that some biological weapons may release more than one pathogen at a time, for example bacteria and virus or more than one strain of bacteria. Therefore, the libraries of mass spectral patterns of immune modulator expression should contain profiles of biological samples taken from animals infected with various combinations of pathogens. The same pattern comparison analysis can be used to obtain a diagnosis.

Based on these observations and results, a new method for diagnosing pathogen infections has been discovered, based on determining a library of mass spectral patterns of expression of immune system modulators (cytokines and chemokines) in biological samples taken from animals known to be infected with known pathogens at various times after exposure and infection. The patterns in the library are compared to mass spectral patterns of expression of immune system modulators in the same type of biological samples taken from animals that may have been exposed and infected by a pathogen in the library. A diagnosis of pathogen infection is made if there is a match between a pattern from the potentially infected animal and a pattern in the library.

In one embodiment, the present invention provides a method for diagnosing pathogen infection in a potentially infected animal using mass spectrometry using a gridless mass spectrometer, that includes determining a mass spectrometry library of patterns of expression for multiple molecular markers of infection in infected samples taken from an animal infected with a known pathogen, each pattern being associated with the known pathogen at a particular post-exposure time following exposure of the animal to the known pathogen; obtaining an analysis sample from a different animal potentially infected with the pathogen; placing the analysis sample on a substratum suitable for use in a mass spectrometer; saturating the analysis sample with matrix material; allowing the analysis sample to dry; processing the analysis sample in the gridless mass spectrometer to obtain a mass spectrograph for the analysis sample; and determining whether the mass spectrograph for the analysis sample includes a first pattern from the library of patterns. If it is determined that the mass spectrograph for the analysis sample includes the first pattern, then generating a diagnosis that the different animal is infected by the known pathogen. The method can also include putting in the library, patterns of expression for multiple molecular marker of infections in infected samples taken from multiple animals infected with a respective plurality of known pathogens, each pattern being associated with a particular pathogen of the plurality of known pathogens at a particular post-exposure time following exposure of a respective animal of the plurality of animals to the particular pathogen; and where the known pathogen is a first pathogen among the known pathogens, which first pathogen is associated with the first pattern in the library of patterns. If it is determined that the mass spectrograph for the analysis sample includes the first pattern, then generating the diagnosis that the different animal is infected by the known pathogen.

Gridless mass spectrometry must be used to analyze the samples, operated in a linear or reflectron mode, with infra red or ultra violet lasers. PSD can be used to optimize resolution of molecules have the same or nearly identical mass.

Computerized Diagnosis

Computer programs can be written to automate diagnosis by comparing patterns stored in the mass spectra library of cytokine expression in a given biological sample at any given time after exposure to known pathogens, and patterns of cytokine expression in “unknown” biological samples taken at similar times following exposure to obtain a diagnosis. As was discussed earlier, mass spectral analysis is not limited to cytokines and chemokines; it can be used to detect the presence of any of other molecular marker of infection that can be resolved in the spectra and verified by comparison to known samples of the marker, including exhaled DNA from pathogens, host response molecules, arachidonic acid metabolites and pathogen-encoded proteins. Because mass spectral analysis of a sample is very fast, on the order of microseconds per sample, the rate limiting part of the diagnosis is obtaining the biological samples and introducing them into the mass spectrometer, not the analysis or diagnostic comparison of patterns of expression of the markers of infection. The more markers of infection that can be analyzed, the higher the probability of a positive diagnosis. Because expression of immune modulators and molecular markers of infection usually changes with time after infection, it is important for a positive diagnosis that the library contain mass spectral patterns of these molecules at many different times following exposure to a known pathogen(s). Again, if the pathogen(s) that were released in a terrorist attack using biological weapons, or that are causing an epidemic, has been identified, diagnosis is simplified because this information can be fed into the computer so that it is taken into consideration in the comparative pattern analysis.

One way of approaching the diagnosis is to ask a series of questions of increasing specificity. The first question is whether or not there has been an infection. To answer this, the computer analysis of the library of expression patterns collected from samples with known infections is analyzed to identify common markers of infection that are expressed at given times following exposure. The second question would be whether the infection is viral, bacterial, parasitic, fungal, etc. If the infection is bacterial, is it gram positive or gram negative? Again, the key is identification of patterns of expression of common markers at given times following initial exposure in cells known to be infected with pathogens (infection by known viruses, bacteria, parasites, fungi, etc.) There may not be a unique match between the mass spectral patterns in the unknown samples and the known infected samples, but by narrowing the diagnosis, the decision of whether to treat or not to treat the potential victim is simplified. Further, knowing whether the bacterium, for example, is gram-positive or gram-negative dictates which antibiotics should be used. Computer programs are easily designed to ask these types of questions and make the comparison of mass spectral patterns.

Since there are so many cytokines, over 200 have now been identified, another embodiment of the present invention is determining from looking at the library of mass spectral patterns, which cytokines are the most relevant to diagnosing infection by one or more known pathogens. The diagnosis can be simplified by searching for those cytokines that are most often changed in known pathogen infected samples in order to make the diagnosis.

Stress may also cause changes in cytokine expression and the expression of other immunomodulators as enumerated herein. Therefore, changes in expression of immune modulators during stress should be analyzed since these changes will most likely occur in people who know they have been or may have been exposed to a bioterrorist attack. The diagnosis should be not be based on markers of infection that are similarly elevated by extreme stress or anxiety.

TABLE 3

| Organism, hour of harvest | IL-1 alpha | IL-1 beta | TNF-alpha** | IL-6 | IL-2 | IFN-gamma |
|---|---|---|---|---|---|---|
| PBS, 1.5 | negative | negative | negative | negative | negative | negative |
| PBS, 3 | negative | negative | negative | negative | negative | negative |
| PBS, 6 | negative | negative | negative | negative | negative | negative |
| LPS, 1.5 | negative | negative | POSITIVE | negative | negative | negative |
| LPS, 3 | POSITIVE | POSITIVE | POSITIVE | POSITIVE | negative | negative |
| LPS, 6 | POSITIVE | POSITIVE | POSITIVE | POSITIVE | negative | negative |
| Bacillus anthracis spores, 1.5 | negative | negative | negative | negative | negative | negative |
| Bacillus anthracis spores, 3 | negative | negative | negative | negative | negative | negative |
| Bacillus anthracis spores, 6 | POSITIVE | POSITIVE | negative | negative | negative | negative |
| Bacillus globigii spores, 1.5 | negative | negative | negative | negative | negative | negative |
| Bacillus globigii spores, 3 | negative | negative | negative | negative | negative | negative |
| Bacillus globigii spores, 6 | negative | negative | negative | negative | negative | negative |
| Yersinia pseudotuberculosis, 1.5 | negative | negative | negative | negative | negative | negative |
| Yersinia pseudotuberculosis, 3 | negative | negative | negative | negative | negative | negative |
| Yersinia pseudotuberculosis, 6 | negative | negative | POSITIVE | negative | negative | negative |
| Yersinia kristensenii, 1.5 | negative | POSITIVE | POSITIVE | negative | negative | negative |
| Yersinia kristensenii, 3 | POSITIVE | POSITIVE | POSITIVE | negative | negative | negative |
| Yersinia kristensenii, 6 | POSITIVE | POSITIVE | POSITIVE | POSITIVE | negative | negative |
| Staphylococcus aureus, 1.5 | negative | negative | negative | negative | negative | negative |
| Staphylococcus aureus, 3 | negative | negative | negative | negative | negative | negative |
| Staphylococcus aureus, 6 | negative | negative | POSITIVE | negative | negative | negative |
| Staphylococcus epidermidis, 1.5 | negative | POSITIVE | negative | negative | negative | negative |
| Staphylococcus epidermidis, 3 | negative | POSITIVE | negative | negative | negative | negative |
| Staphylococcus epidermidis, 6 | negative | POSITIVE | POSITIVE | negative | negative | negative |
| Haemophilus influenza, 1.5 | negative | POSITIVE | POSITIVE | negative | negative | negative |
| Haemophilus influenza, 3 | POSITIVE | POSITIVE | POSITIVE | negative | negative | negative |
| Haemophilus influenza, 6 | negative | POSITIVE | POSITIVE | negative | negative | negative |

**For TNF alpha only, PBS results showed signs of higher than Tissue Culture Media only.
All reported values represent positives relative to PBS control

Exhaled Breath Concentration Collection

Many studies have verified the presence of protein in exhaled breath condensate taken from humans, including many different cytokines. It has also been shown that expression of some cytokines and chemokines changes when an animal has a respiratory disease, including infection. As was discussed above, chronic respiratory diseases and smoking can cause an atypical elevation in certain cytokines that could complicate diagnosis. Therefore, people with these conditions should be identified and may need to be handled separately. By identifying and comparing expression of many different cytokines, chemokines and especially pathogen-encoded proteins, one can eliminate reliance on changes in markers that are elevated in chronic respiratory diseases or smoking for diagnosis in this special group of potential victims.

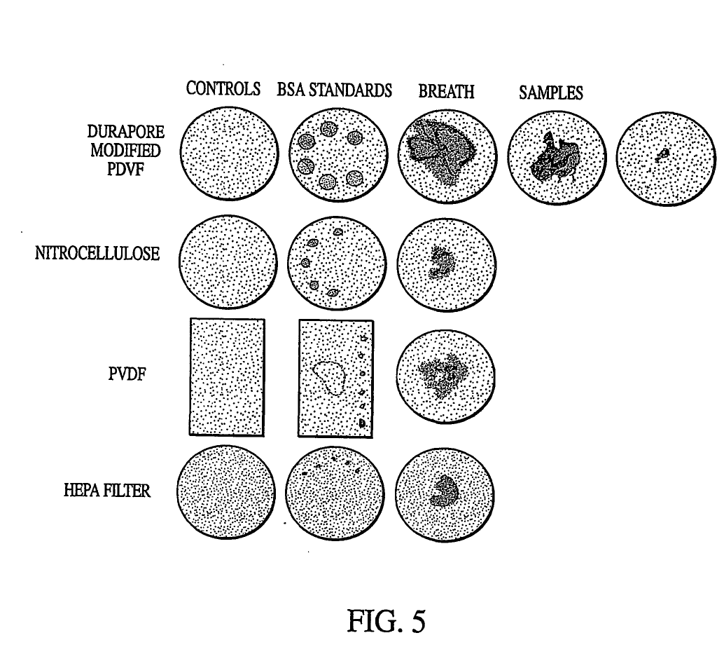

Breath samples were collected on various filters suitable for use in mass spectrometers, including Durapore Modified PDVF (Millipore), nitrocellulose, polyvinylidene fluoride (PVDF), and Hepa filter material. The results of analysis of protein samples of controls, BSA standards diluted in phosphate buffered saline (PBS), and breath from human volunteers is presented in FIG. 5 and in Table 4.

It was discovered that only two deep breaths were required to obtain detectable protein in exhaled condensate. Thus, using breath condensate to analyze changes in infection markers is not only feasible, but also practical. Durapore membranes provided the most reproducible recovery of protein from filters. The filter impregnated with protein condensate and with the sinapinic acid matrix will be introduced into the MALDI mass spectrometer for analysis.

The ideal integrated collection device would permit collection and condensation of breath vapor on filters or tubes. The samples would be manually placed into a commercial MALDI-TOF, for example a miniaturized TOF. Such a spectrometer, known as the TINY TOF, is currently under development and testing at the Johns Hopkins University Applied Physics Laboratory. The TINY TOF will also permit rapid high-resolution spectral analysis of the exhaled air condensates and on-line laser fragmentation of small molecular weight ions (<30 Kda). Bench top MALDI-TOF instruments are currently available commercially for compilation of unique spectra signatures (manufactured by, e.g., Kratos and Bruker).

Routine experimentation will determine whether simple air-cooled condensers will optimize collection of exhaled water vapor, and whether concentration or filtration will be required to obtain the necessary sensitivity for diagnosis. One collection device is comprised of a low cost, single-use polycarbonate breath restrictor and filter device smaller than 6 inches overall. This device is connected to a temperature-regulated manifold for chilling and condensation of breath vapor. The collector in turn is attached to a low volume vacuum pump to optimize the rapid chilling which may be needed to preserve sample integrity. A solenoid valve would permit breath collection under normal unpressurized conditions and then apply backpressure as required to collect breath under pressurized conditions for accurate analysis based on breath volume. A small nebulizer and aerosol pump could be connected into the same device through a port near the filter. The nebulizer and pump would serve to evenly deposit matrix from a bulk source during or immediately following sample collection. This would permit the same device to be used for sample collection and on-line sample preparation for mass spectrometry. In another embodiment, the sample collector is connected to an on-line NO analyzer for real time NO determination.

The breakaway collection device can be disassembled for access to the collected breath condensate on the filter; collection devices should be disposable. The filter is manually placed into the miniaturized MALDI-TOF mass spectrometer for targeted spectral analysis of the breath condensates using this proposed prototype. Ambulances can be equipped with full size linear mass spectrometers to mobilize this technology to sites where potential victims can be screened.

In the foregoing specification, the invention has been described with reference to specific embodiments thereof. It will, however, be evident that various modifications and changes may be made to the inventions without departing from the broader spirit and scope of the invention. Embodiments of the present inventions are described in further detail below, but are not limited by the examples.

### EXAMPLES

### Example 1

Materials and Methods for Cell Culture and Cytokine Analysis in Raw Cells

Purified murine cytokines were obtained from various manufacturers (Sigma, and RND Systems). Expressed cytokines were obtained from the supernatants of murine RAW 264.7 (RAW cells) macrophagocytic cells that were either controls or exposed to one or more pathogens. RAW cells were obtained from the American Tissue Culture Collection and cultured at 37° C. in 5% carbon dioxide in RPMI 1640 media (Gibco BRL) supplemented with 10% heat inactivated fetal bovine serum albumin (Gibco/Intergen). Exponentially growing RAW cultures were cultured in 6 well plates to 65%-80% confluence.

LPS (Sigma) was used as a positive control in tissue culture assays to induce cytokine expression. Bacteria at an MOI of 1 was added to triplicate cultures for analysis of the supernatant (harvested by collection and centrifugation) 0, 1.5, 3.0 and 6.0 hours following exposure. Expressed cytokines in tissue culture fluids were analyzed by ELISA assay using murine antibodies prior to analysis by MALDI-TOF. Results are expressed as pg/ml of tissue culture fluid.

ELISA analysis was performed using RND systems ELISA kits for Tumor Necrosis Factor alpha (TNFa), Interleukins 1a, 1b, 2, 6 and interferon gamma. ELISAs were performed according to the manufacturer's directions.

Mass spectrometry was performed using a gridless Kratos Axima MALDI-TOF mass spectrometer with and without pulsed extraction. The MALDI-TOF was calibrated with cytochrome C or BSA (bovine serum albumin) in Sinapinic acid matrix. Purified cytokines standards were stored at −70° C. before being reconstituted to equal molar concentrations based on manufacturer's determination of the amount of cytokine. BSA was added by the manufacturer to all cytokine samples. Human sputum was obtained from a donor. Sputum was obtained according to established methods by expectoration. Cytokines were mixed with sputum samples at the reported ratios based on volume.

Neat samples were prepared for MALDI analysis as follows. Sinapinic acid or alpha cyano matrix was used for all analysis. Subsequent experiments with other matrices indicated that sinapinnic acid was the preferred matrix. All pictured analyses were performed with sinapinic acid prepared in 70% acetonitrile (HPLC grade) and 30% acidified water (HPLC Grade). Water is acidified with addition of 0.1% trifluoroacetic acid. 0.3 microliter of matrix is used to coat the substratum. Before fully dried, 0.3 microliter of test material is added to the matrix and 0.3 microliter of additional matrix is added to the test material. The matrix-saturated sample is allowed to dry prior to placing in MALDI-TOF. Spectra were obtained from pure samples and mixtures using the same sample preparation conditions. To obtain standard spectra of pure samples of the immune system modulators being analyzed for comparison purposes, each pure sample was run individually.

The mass spectrometer was periodically recalibrated so that the standard spectra obtained for pure samples of immune system modulators could be used for comparison purposes without redoing the standards. The frequency of calibration depends on the number of samples being run and variability among mass spectrometers.

### EXAMPLE II

Bacterial and Viral Induced Changes in Cytokine Expression in Murine RAW 264.7 (RAW Cells) Macrophagocytic Cells

Triplicate samples of murine RAW 264.7 (RAW cells) macrophagocytic cells were exposed to bacterial agents (gram negative and gram positive), viruses (threat agents and common environmental agents), toxins (fungal and bacterial) and particulate materials (components of biological weapons preparations and respirable particulates) in order to evaluate the sensitivity, specificity and robustness of the cytokine response. Bacteria and unactivated LPS were added to the culture media at a Multiplicity of infection (MOI) of 1, which equals 1 infectious unit per 1 cell. Supernatant was harvested by collection and centrifugation at 0, 1.5, 3.0 and 6.0 hours following exposure.

Bacterial agents: unactivated LPS, Yersinia Pseudotuberculosis, Yersinia kristensenii Bacillus anthracis spores, Staphylococcus aureus, Staphylococcus epidermis, Streptococcus pneumoniae, Haemophilus influenza.

Viral agents: Venezuelan Equine Encephalitis (VEE), Influenza.

MALDI-TOF cytokine signatures were obtained using purified murine cytokines and all spectra of samples of complex mixtures of murine cytokines from supernatants were compared to the spectra of purified murine cytokines and to ELISA analysis of the pure murine cytokines.

Supernatant was harvested by collection and centrifugation 0, 1.5, 3.0 and 6.0 hours following exposure. Mass spectrometry was performed using a Kratos AXIMA MALDI-TOF gridless mass spectrometer in linear mode with ultra violet lasers, with and without pulsed extraction. A matrix of sinapinic acid prepared in 70% acetonitrile HPLC grade) and 30% acidified water (HPLC Grade) was used. The results of analysis are given in Table 3.

### EXAMPLE III

Protein Collection on and Recovery from Various Filter Types

BSA protein diluted in PBS was dropped onto the filters. BSA protein recovery from filters was determined by extracting proteins from filter materials and performing spectrophotometry at 280 nm to look for absorption in addition to performing BCA assay according to the manufacturer's directions (Pierce). Briefly protein was extracted from filters using PBS supplemented with 0.5% Tween 20. For low recovery on some filter materials 70% acetonitrile was also used, however, either no additional recovery of protein was seen or the filter material itself degraded when exposed to acetonitrile. These filters which degraded in the presence of acetonitrile could not be used for UV MALDI but remained under consideration as they could be used of IR MALDI.

TABLE 4

| | | 200 ug/ml | 100 ug/ml | 80 ug/ml | 60 ug/ml | 40 ug/ml | 20 ug/ml | 0 ug/ml |
|---|---|---|---|---|---|---|---|---|
| Durapore HVLP | Recovered | 188.09 | 93.08 | 80.17 | 60.08 | 41.22 | 23.57 | 0.11 |
| | (ug/ml) | | | | | | | |
| | % Recovery | 94% | 93% | 100% | 100% | 102% | 117% | 100% |
| Nitrocellulose | Recovered | 70.36 | 23.69 | 16.75 | 17.87 | 13.05 | 14.4 | 11.71 |
| | (ug/ml) | | | | | | | |
| | % Recovery | 35% | 23% | 21% | 29% | 32% | 72% | 100% |
| PVDF | Recovered | 162.91 | 97.03 | 68.64 | 59.76 | 51.07 | 19.02 | 0.53 |
| | (ug/ml) | | | | | | | |
| | % Recovery | 81% | 97% | 86% | 99% | 127% | 95% | 100% |
| Forward Layer of | Recovered | 112.16 | 2.72 | 32.97 | 14.48 | 18.23 | 9.66 | 0.83 |
| Lifeline | (ug/ml) | | | | | | | |
| Pulmonary Filter | % Recovery | 56% | 3% | 41% | 24% | 45% | 48% | 100% |
| | | 100 ug/ml | 50 ug/ml | 40 ug/ml | 30 ug/ml | 20 ug/ml | 10 ug/ml | 0 ug/ml |
| Whole Lifeline | Recovered | 97.26 | 56.06 | 41.14 | 45.61 | 32.9 | 17.26 | 0.56 |
| Pulmonary Filter | (ug/ml) | | | | | | | |
| | % Recovery | 97% | 112% | 102% | 152% | 106% | 172% | 100% |

Filters were stained with Comassie Blue or with the transient dye Ponceau S. Filters were stained for up to 20 minutes in 20% Comassie Blue and extraneous dye was removed by washing filters 2-3 times in destaining solution (10% acetic acid, 10% methanol). Ponceau S was prepared according to manufacturer's directions. Filters were stained in Ponceau S for up to 5 minutes and destained in acidified deionized distilled water.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

What is claimed is:

1. A method for diagnosing pathogen infection in a potentially infected animal using mass spectrometry, comprising the steps of:
a. using a gridless mass spectrometer, determining a mass spectrometry library of patterns of expression for multiple molecular markers of infection in infected samples taken from an animal infected with a known pathogen, each pattern being associated with the known pathogen at a particular post-exposure time following exposure of the animal to the known pathogen;
b. obtaining an analysis sample from a different animal potentially infected with the pathogen;
c. placing the analysis sample on a substratum suitable for use in a mass spectrometer;
d. saturating the analysis sample with matrix material;
e. allowing the analysis sample to dry after step d;
f. processing the analysis sample in the gridless mass spectrometer to obtain a mass spectrograph for the analysis sample;
g. determining whether the mass spectrograph for the analysis sample includes a first pattern from the library of patterns; and
h. if it is determined that the mass spectrograph for the analysis sample includes the first pattern, then generating a diagnosis that the different animal is infected by the known pathogen.

2. A method as recited in claim 1, wherein:
step a further comprises including in the library, patterns of expression for multiple molecular marker of infections in infected samples taken from a plurality of animals infected with a respective plurality of known pathogens, each pattern being associated with a particular pathogen of the plurality of known pathogens at a particular post-exposure time following exposure of a respective animal of the plurality of animals to the particular pathogen; and
in step h the known pathogen is a first pathogen of the plurality of known pathogens, which first pathogen is associated with the first pattern in the library of patterns.

3. A method as recited in claim 1, step h further comprising, if it is determined that the mass spectrograph for the analysis sample includes the first pattern, then generating the diagnosis that the different animal is infected by the known pathogen at an infection time determined based on a particular post-exposure time associated with the first pattern and an analysis sample time when step b is performed.

4. The method of claim 1, wherein the mass spectrometer is a matrix assisted laser desorption/ionization (MALDI) mass spectrometer.

5. The method of claim 1, wherein the mass spectrograph is obtained with pulsed extraction.

6. The method of claim 1, wherein the matrix is a sinapinic acid matrix.

7. The method of claim 1, wherein step (g) further comprises the step of employing post-source decay (PSD) analysis.

8. The method of claim 1, wherein the molecular marker of infection is an immune system modulator.

9. The method of claim 8, wherein the immune system modulator has a molecular mass of about 30 kilo Daltons or less.

10. The method of claim 8, wherein the immune system modulator is selected from the group comprising cytokines, chemokines, virokines, pathogen-encoded immune modulator receptors and leukotrienes.

11. The method of claim 1, wherein the mass spectrometer uses either an infrared laser or an ultra violet laser.

12. The method of claim 1, wherein the pathogen is a bacterium.

13. The method of claim 1, wherein the pathogen is a virus.

14. The method of claim 1, wherein the pathogen is a parasite.

15. The method of claim 1, wherein the pathogen is a fungus.

16. The method of claim 1, wherein the mass spectrometer is a operated in a linear mode.

17. The method of claim 1, wherein the mass spectrometer is operated in a reflectron mode.

18. The method of claim 1, wherein the mass spectrograph is obtained using post source decay analysis.

19. The method of claim 1, wherein the molecular infection marker is a peptide.

20. The method of claim 1, wherein the molecular infection marker is a prion.

21. The method of claim 1, wherein the molecular infection marker is a pathogen-encoded molecule or fragment thereof.

22. The method of claim 1, wherein the molecular infection marker is a pathogen-encoded DNA fragment.
